(19) United States
(12) Patent Application Publication
(10) Pub. No.: US 2007/0292511 A1
(43) Pub. Date: Dec. 20, 2007

(54) Duloxetine hydrochloride delayed release formulations

(76) Inventors: Gershon Kolatkar, Petah Tiqva (IL); Erela Zisman, Rishon Le Zion (IL)

Correspondence Address: KENYON & KENYON LLP, ONE BROADWAY, NEW YORK, NY, 10004, US

(21) Appl. No.: 11/805,395

(22) Filed: May 22, 2007

Related U.S. Application Data
(60) Provisional application No. 60/802,849, filed on May 22, 2006.

(51) Int. Cl. A61K 9/24; A61K 31/381; A61P 25/24

(52) U.S. Cl. 424/471; 514/448

(57) ABSTRACT

Delayed release formulations of duloxetine hydrochloride and methods for its manufacture are described. A preferred formulation includes an inert core, a drug layer comprising duloxetine hydrochloride, a separating layer and an enteric layer comprising at least one of methacrylic acid copolymer and hydroxypropyl methyl cellulose phthalate.

### CROSS-REFERENCE TO RELATED APPLICATIONS

This application claims the benefit of U.S. provisional application No. 60/802,849, filed May 22, 2006, herein incorporated by reference.

### FIELD OF THE INVENTION

The invention encompasses duloxetine hydrochloride delayed release formulations and methods for their manufacture.

### BACKGROUND OF THE INVENTION

Duloxetine hydrochloride is a selective serotonin and norepinephrine reuptake inhibitor (“SSRI”), having the chemical name (+)-(S)—N-methyl-γ-(1-naphthyloxy)-2-thiophenepropylamine hydrochloride, a molecular formula of C₁₈H₁₉NOS.HCl, and a molecular weight of 333.88. The chemical structure of duloxetine hydrochloride may be represented by Formula I.

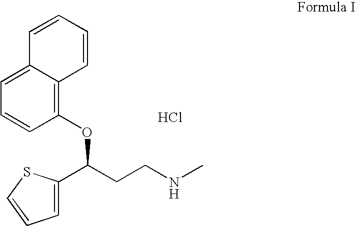

Duloxetine hydrochloride is disclosed in European Publication No. 273658, and is currently marketed by Eli Lilly for the treatment of major depressive disorder under the trade name CYMBALTA® as 20, 30, and 60 mg delayed release enteric-coated capsules. CYMBALTA® tablets reportedly contain duloxetine hydrochloride and the inactive ingredients FD&C Blue No. 2, gelatin, hypromellose, hydroxypropyl methylcellulose acetate succinate, sodium lauryl sulfate, sucrose, sugar spheres, talc, titanium dioxide, triethyl citrate, and, optionally, iron oxide yellow.

U.S. Pat. No. 5,508,276 (“the '276 patent”) discloses a delayed release duloxetine formulation in the form of an enteric duloxetine pellet. The disclosed enteric coating layer contains an enteric polymer having only a small number of carboxylic acid groups per repeating unit. Hydroxypropyl methylcellulose acetate succinate (“HPMCAS”) is disclosed as the preferred enteric polymer. When HPMCAS is applied in the form of a suspension, the '276 patent discloses that it is advisable to cool the suspension below 20° C. before application, as well as to use tubing with a small diameter and to cool the tubing and nozzle of the spray-drier. When HPMCAS is applied in the form of an aqueous solution, the '276 patent discloses that the HPMCAS should be neutralized, for example, with ammonia to facilitate its dissolution. The '276 patent also discloses that duloxetine was found to react with many enteric coatings to form a slowly soluble or insoluble coating. This may lead to a disadvantageous drug-releasing profile and/or low bioavailability. The '276 patent also discloses that the enteric pharmaceutical formulations are manufactured in such a way that the product passes unchanged through the stomach of the patient, and dissolves and releases the active ingredient quickly when it leaves the stomach and enters the small intestine. This is accomplished by enclosing the active ingredient in the inner part of the tablet or pellet in a film or envelope, the “enteric coating”, which is insoluble in acid environments, such as the stomach, but is soluble in near-neutral environments such as the small intestine.

Delayed release formulations are advantageous, as they prevent exposure of an acid sensitive active pharmaceutical ingredient (“API”) to the acidic environment of a patient's stomach, preventing degradation of the API and/or irritation of the patient's stomach. Thus, additional delayed release formulations of duloxetine hydrochloride would be advantageous. The present invention provides such a delayed formulation of duloxetine hydrochloride.

### SUMMARY OF THE INVENTION

The invention encompasses a duloxetine hydrochloride delayed release formulation comprising an inert core, a drug layer comprising duloxetine hydrochloride, a separating layer, an enteric layer comprising at least one of a methacrylic acid copolymer and hydroxypropyl methyl cellulose phthalate, and, optionally, a finish layer. Preferably, the inert core comprises sugar spheres or pellets of microcrystalline cellulose.

Preferably, the drug layer further comprises one or more pharmaceutically acceptable excipients. More preferably, the excipients are selected from binders, glidants, coating agents, and anti-static agents. Most preferably, the excipients are selected from sucrose, povidone, colloidal silicon dioxide, hypromellose, and talc. A particularly preferred drug layer comprises duloxetine hydrochloride, sucrose, povidone, colloidal silicon dioxide, and hypromellose. The drug layer is preferably present in an amount of about 40 percent to about 90 percent by weight of the formulation. More preferably, the drug layer is present in an amount of about 50 percent to about 75 percent by weight of the formulation.

The separating layer preferably comprises a coating agent and, optionally, one or more additional pharmaceutically acceptable excipients. Preferably, the excipients are selected from diluents, anti-adherents, and thickening agents. More preferably, the excipients are selected from sucrose, talc, povidone, and colloidal silicon dioxide. A particularly preferred separating layer comprises hypromellose, titanium dioxide, iron oxide, sucrose, and talc. The separating layer is preferably present in an amount of about 8 percent to about 60 percent by weight of the formulation. More preferably, the separating layer is present in an amount of about 15 percent to about 45 percent by weight of the formulation.

In addition to the methacrylic acid copolymer and/or hydroxypropyl methyl cellulose phthalate, the enteric layer preferably further comprises one or more pharmaceutically acceptable excipients. Preferably, the excipients are selected from glidants and plasticizers. More preferably, the excipients are selected from talc and triethyl citrate. The enteric layer is preferably present in an amount of about 5 percent to about 40 percent by weight of the formulation. More preferably, the enteric layer is present in an amount of about 10 percent to about 30 percent by weight of the formulation.

The optional finish layer may comprise a coating agent and, optionally, one or more additional pharmaceutically acceptable excipients. Preferably, the excipients are selected from thickening agents, glidants, and coloring agents. Preferably, the excipients are selected from talc, colloidal silicon dioxide, and titanium dioxide. A particularly preferred finish layer comprises hypromellose, talc, colloidal silicon dioxide, and titanium dioxide. The finish layer is preferably present in an amount of about 1 percent to about 15 percent by weight of the formulation. More preferably, the finish layer is present in an amount of about 2 percent to about 10 percent by weight of the formulation.

The invention also encompasses a process for preparing the duloxetine hydrochloride delayed release formulation of the invention. The process preferably comprises coating a core in successive steps with a drug layer comprising duloxetine hydrochloride; a separating layer; an enteric layer comprising at least one of a methacrylic acid copolymer and hydroxypropyl methyl cellulose phthalate; and, then, optionally, a finish layer.

More preferably, each of the drug layer, separating layer, enteric layer, and optional finish layer are applied from a solution and/or suspension of the components of each layer. Most preferably, each layer is applied by spraying the core or previously formed layer with an appropriate solution and/or suspension that will form the desired layer.

For example, a delayed release duloxetine hydrochloride formulation in accordance with the invention may be formed by coating an inert core in successive steps with a solution comprising duloxetine hydrochloride to form the drug layer, a suspension of components that will form the separating layer, a suspension of at least one of a methacrylic acid copolymer and hydroxypropyl methyl cellulose phthalate to form the enteric layer, and, optionally, a suspension of components that will form the finish layer, wherein the core is preferably dried between each coating step.

A duloxetine hydrochloride delayed release formulation in accordance with the invention may be prepared in a preferred process that comprises coating an inert core with a solution comprising duloxetine hydrochloride and, optionally, one or more excipients, such as sucrose, povidone, colloidal silicon dioxide, and hypromellose, in a solvent or mixture of solvents, such as water, ethanol, and mixtures thereof, where the solvent is most preferably an 80:20 mixture of water and ethanol, and preferably drying the core. The duloxetine hydrochloride coated core is then coated with a suspension comprising a coating agent and, optionally, one or more additional pharmaceutically acceptable excipients, such as diluents, anti-adherents, or thickening agents, where the suspension most preferably comprises hypromellose, titanium dioxide, iron oxide, sucrose, and talc in water, thereby forming a separating layer, which is then preferably dried. The duloxetine hydrochloride and separating layer coated core is then coated with at least one of a methacrylic acid copolymer and hydroxypropyl methyl cellulose phthalate and, optionally, one or more pharmaceutically acceptable excipients, such as hypromellose, titanium dioxide, iron oxide, sucrose, triethyl citrate, and talc, in a solvent, such as water, and dried, thereby forming an enteric coating on the core coated with duloxetine hydrochloride and separating layer.

Where a finish layer is desired, the process of the invention preferably further comprises coating the core coated with duloxetine hydrochloride, separating layer, and enteric layer with a suspension of a coating agent and, optionally, one or more additional pharmaceutically acceptable excipients, such as thickening agents, glidants, or coloring agents, where the suspension most preferably comprises hypromellose, talc, colloidal silicon dioxide, and titanium dioxide in water, and drying the coating, thereby forming the finish layer.

The invention also encompasses a solid pharmaceutical dosage form comprising the duloxetine hydrochloride delayed release formulation. Preferably, the solid pharmaceutical dosage form is a capsule.

The invention also encompasses a method for the treatment of depression comprising administering the duloxetine hydrochloride delayed release formulation of the invention to a patient in need thereof.

### DETAILED DESCRIPTION OF THE INVENTION

The invention encompasses a duloxetine hydrochloride delayed release formulation with an enteric layer comprising, for example, methacrylic acid copolymer and/or hydroxypropyl methyl cellulose phthalate. Use of an enteric layer comprising methacrylic acid copolymer and/or hydroxypropyl methyl cellulose phthalate, for example, generally has several advantages over HPMCAS. For example, methacrylic acid copolymer and hydroxypropyl methyl cellulose phthalate are more suitable for use on an industrial scale because they can be handled at room temperature with standard equipment. In addition, no neutralization of these polymers is necessary during processing. Further, using methacrylic acid copolymer and/or hydroxypropyl methyl cellulose phthalate, as opposed to HPMCAS, in the enteric coat of preferred embodiments allows for a duloxetine formulation that has a good releasing profile and good bioavailability.

The invention encompasses a duloxetine hydrochloride delayed release formulation comprising: (a) an inert core; (b) a drug layer comprising duloxetine hydrochloride; (c) a separating layer; (d) an enteric layer comprising at least one of a methacrylic acid copolymer and hydroxypropyl methyl cellulose phthalate; and, optionally, (e) a finish layer.

The core may comprise any inert material or mixture of materials known to one of skill in the art of drug formulation for use as cores that does not interact adversely with duloxetine hydrochloride. Preferably, the core comprises sugar spheres or pellets of microcrystalline cellulose NF. The core is preferably present in an amount of not more than about 50 percent by weight of the formulation. More preferably, the core is present in an amount of not more than about 40 percent by weight of the formulation. Preferably, the core is present in a weight ratio of about 1:1 to about 2.5:1 relative to the drug layer.

Preferably, the drug layer comprises duloxetine hydrochloride and one or more pharmaceutically acceptable excipients. The pharmaceutically acceptable excipients may include excipients commonly used in pharmaceutical formulations that do not interact adversely with duloxetine hydrochloride. Preferably, the pharmaceutically acceptable excipients are selected from diluents, binders, glidants, coating agents, and anti-static agents. More preferably, the pharmaceutically acceptable excipients are selected from sucrose, povidone, colloidal silicon dioxide, hypromellose, and talc USP. The drug layer is preferably present in an amount of about 40 percent to about 90 percent by weight of the formulation. More preferably, the drug layer is present in an amount of about 50 percent to about 75 percent by weight of the formulation. Preferably, the drug layer is present in a weight ratio of about 0.5:1 to about 2:1 relative to the separating layer.

A particularly preferred drug layer comprises duloxetine hydrochloride, sugar spheres, povidone USP, (PVP K-30), AEROSIL® 200 (colloidal silicon dioxide NF), and talc USP. More preferably, the drug layer comprises about 10-70% duloxetine hydrochloride, about 20-80% sugar spheres, about 1-30% povidone USP (PVP K-30), about 1-10% AEROSIL® 200 (colloidal silicon dioxide NF), and about 1-20% talc USP, wherein the percentages are by weight of the drug layer.

The separating layer preferably performs one or more of the following functions: providing a smooth base for the application of the enteric layer, prolonging the formulation's resistance to the acidic environment of the stomach, improving stability of the formulation by inhibiting interaction between the duloxetine hydrochloride and the enteric layer, or improving storage stability of the formulation by protecting the duloxetine hydrochloride from exposure to light. The separating layer preferably comprises a coating agent and, optionally, one or more additional pharmaceutically acceptable excipients. Preferably, the coating agent is selected from at least one of OPADRY® and hydroxypropyl methyl cellulose. OPADRY®, available from Colorcon (West Point, Pa.), contains hydroxypropyl cellulose, hypromellose, titanium dioxide, and iron oxide. One of skill in the art would recognize that a mixture of these ingredients can be substituted for the commercially available pre-mixed OPADRY® formulation without departing from the scope of the invention.

The additional pharmaceutically acceptable excipients may include excipients commonly used in pharmaceutical formulations that do not interact adversely with duloxetine hydrochloride. Preferably, the additional pharmaceutically acceptable excipients are selected from diluents, anti-adherents, and thickening agents. More preferably, the additional pharmaceutically acceptable excipients are selected from sucrose, talc, povidone USP (PVP K-30), and colloidal silicon dioxide (AEROSIL® 200). The separating layer is preferably present in an amount of about 8 percent to about 60 percent by weight of the formulation. More preferably, the separating layer is present in an amount of about 15 percent to about 45 percent by weight of the formulation. Preferably, the separating layer is present in a weight ratio of about 0.5:1 to about 3:1 relative to the enteric layer.

A particularly preferred separating layer comprises OPADRY® white 39A28677, PHARMACOAT® 606 (hypromellose USP), sucrose NF, and talc USP. More preferably, the separating layer comprises about 10-70% OPADRY® white 39A28677, about 1-15% PHARMACOAT® 606 (hypromellose USP), about 5-60% sucrose NF, and about 20-75% talc USP, wherein the percentages are by weight of the separating layer.

The enteric layer is applied to accomplish delayed release of the duloxetine hydrochloride primarily in the small intestine. Preferably, the enteric layer is substantially insoluble in acidic environments, such as the stomach, but is soluble in near-neutral environments, such as the small intestine. Thus, the formulation remains in tact as it passes through the acid environment of the stomach, but dissolves and releases the duloxetine hydrochloride once it passes into the near-neutral environment of the small intestine. The enteric layer preferably contains a polymer that dissolves at a pH of above about 5.5. The enteric layer comprises hydroxypropyl methyl cellulose phthalate and/or a methacrylic acid copolymer, such as EUDRAGIT® methacrylic acid copolymer dispersion, e.g., EUDRAGIT® L30D55, available from Degussa, Düsseldorf, Germany, and, optionally, one or more additional pharmaceutically acceptable excipients. The additional pharmaceutically acceptable excipients may include excipients commonly used in pharmaceutical formulations for use in enteric layers that do not interact adversely with duloxetine hydrochloride. Preferably, the additional pharmaceutically acceptable excipients are selected from glidants and plasticizers. More preferably, the additional pharmaceutically acceptable excipients are selected from talc and triethyl citrate. The enteric layer is preferably present in an amount of about 5 percent to about 40 percent by weight of the formulation. More preferably, the enteric layer is present in an amount of about 10 percent to about 30 percent by weight of the formulation. Preferably, the enteric layer is present in a weight ratio of about 6:1 to about 12:1 relative to the finish layer.

A particularly preferred enteric layer comprises EUDRAGIT® L30D55 (30% aqueous dispersion), triethyl citrate NF, and talc USP. More preferably, the enteric layer comprises about 5-70% EUDRAGIT® L30D55 (30% aqueous dispersion), about 5-30% triethyl citrate NF, and about 10-50% talc USP, wherein the percentages are by weight of the enteric layer.

The optional finish layer is preferably applied to aid in the handling of the formulation. The enteric coating has some electrostatic force, which may result in the formulation sticking to the packaging; the finish layer prevents the enteric coating from coming into contact with the packaging, thereby avoiding this problem. The optional finish layer preferably comprises a coating agent and, optionally, one or more additional pharmaceutically acceptable excipients. Preferably, the coating agent is hypromellose. The additional pharmaceutically acceptable excipients may include excipients commonly used in pharmaceutical formulations for use in finish layers or coatings. Preferably, the additional pharmaceutically acceptable excipients are selected from thickening agents, glidants, and coloring agents. More preferably, the additional pharmaceutically acceptable excipients are selected from talc, colloidal silicon dioxide, and titanium dioxide. The finish layer is preferably present in an amount of about 1 percent to about 15 percent by weight of the formulation. More preferably, the finish layer is present in an amount of about 2 percent to about 10 percent by weight of the formulation.

A particularly preferred finish layer comprises talc USP, PHARMACOAT® 603 (hypromellose), and AEROSIL® 200 (colloidal silicon dioxide NF). More preferably, the finish layer comprises about 5-50% talc USP, about 5-50% PHARMACOAT® 603 (hypromellose), and about 5-30% AEROSIL® 200 (colloidal silicon dioxide NF), wherein the percentages are by weight of the finish layer.

The invention also encompasses a process for preparing the duloxetine hydrochloride delayed release formulation, comprising coating a core in succession with a drug layer comprising duloxetine hydrochloride; a separating layer; an enteric layer comprising at least one of hydroxypropyl methyl cellulose phthalate and a methacrylic acid copolymer; and then, optionally, a finish layer. Preferably, each layer is applied in the form of a suspension and/or a solution, and, more preferably, each layer is spray coated. Preferably, each layer is dried prior to the application of the next successive coating.

The solution of drug layer may be prepared by combining the components of the drug layer with water or a mixture of water and alcohol. Preferably, the components of the drug layer are combined with a mixture of water and ethanol. More preferably, the drug layer components are combined with an 80:20 mixture of purified water:ethanol. Most preferably, the ethanol is 95 percent ethanol. The purified water preferably meets the specifications recited in the U.S. Pharmacopeia (29th ed. 2005).

The suspensions of the components of the separating layer, enteric layer, and finish layer are preferably prepared by combining the constituents of the respective layers with water, which is preferably purified water.

Each layer of the formulation may be formed by any method known to one of ordinary skill in the art. For example, the each layer may be applied to the core with the above-described solutions or suspensions by any conventional technique known to one of ordinary skill in the art. Preferably, the coating layers are formed by spraying the solutions or suspensions onto the core.

Preferably, solutions or suspensions are sprayed onto the core, while mixing, through a nozzle of about 1 to about 1.2 mm. Preferably, the solutions or suspensions are sprayed with an atomizing air pressure of about 2 to about 2.5 bar. Preferably, the inlet air temperature is about 30° C. to about 60° C. Preferably, the outlet air temperature is about 25° C. to about 50° C. Preferably, the flap is about 80 to about 100 m³/hr. Preferably, the spray rate is about 5 to about 10 g/min.

Preferably, the core is dried between coatings by placing the core in a fluid bed dryer. More preferably, the core is dried at a temperature of about 402 C. Preferably, the coated core is dried for about 5 minutes to about 120 minutes.

A particularly preferred process of the invention for preparing the duloxetine hydrochloride delayed release formulation of the invention comprises: (a) providing an inert core; (b) coating the core with a solution of duloxetine hydrochloride, sucrose, povidone, colloidal silicon dioxide, and hypromellose in a mixture of water and ethanol; (c) optionally drying the core; (d) coating the previously coated core with a suspension of hydroxypropyl cellulose, hypromellose, titanium dioxide, and iron oxide, sucrose, and talc in water; (e) optionally drying the core; (f) coating the previously coated core with a suspension of methacrylic acid co-polymer, talc, and triethyl citrate in water; and (g) optionally drying the core.

The process may further comprise the steps of: (h) coating the previously coated core with a suspension of hypromellose, talc, colloidal silicon dioxide, and titanium dioxide in water; and (i) optionally drying the core.

Once prepared, the duloxetine hydrochloride delayed release formulation may be packaged into a solid pharmaceutical dosage form, such as a tablet or capsule. Preferably, the formulation is filled into a capsule.

In accordance with the invention, depression may be treated in a method comprising administering the duloxetine hydrochloride delayed release formulation to a patient in need thereof.

The following non-limiting examples are merely illustrative of the preferred embodiments of the present invention, and are not to be construed as limiting the invention, the scope of which is defined by the appended claims.

### EXAMPLES

High Performance Liquid Chromatography

The presence and amount of duloxetine hydrochloride impurities in tablets of duloxetine hydrochloride were analyzed by HPLC under the following conditions:
Column: Inertsil ODS-3, 3 micron, 4.6×150 mm
Mobile Phase: Solution A: Buffer solution: acetonitrile (80:20)
   Solution B: Buffer solution: acetonitrile (25:75)
Column Temperature: 40° C.
Detector: UV at 290 nm

### Example 1

### Preparation of a Duloxetine Hydrochloride Delayed Release Capsule Containing an Enteric Layer of Methacrylic Acid Co-Polymer

Part I—Core

Sugar spheres were obtained, and placed in a fluid bed dryer. The average diameter of the sugar spheres was 850-1000 microns.

Part II—Drug Layer

Sucrose, povidone, duloxetine hydrochloride, colloidal silicon dioxide, and hypromellose were mixed with a solution of 85 percent purified water and 15 percent ethanol in a mixer until the solids were fully dissolved.

The resulting solution was sprayed, while mixing, onto the sugar spheres in the fluid bed dryer through a 1 mm nozzle at an atomizing air pressure of 2.5 bar. The inlet air temperature was 60° C., the outlet air temperature was 48° C., the flap was 100 m³/hr, and the spray rate was 5 to 10 g/min. The coated sugar spheres were then dried in the fluid bed dryer for an additional 5 minutes at 40° C. to form drug-coated pellets.

Part III—Separating Layer

Sucrose, OPADRY® 39A28677, and hypromellose were mixed in purified water in a mixer until fully dissolved to form a solution. Talc was mixed in purified water in a homogenizer for 30 minutes, and the resulting mixture of talc and water was added to the solution in the mixer. The resulting mixture was mixed for 15 minutes.

The resulting suspension was sieved, and then sprayed onto the drug-coated pellets in the fluid bed dryer. The suspension was sprayed while mixing through a 1.2 mm nozzle at an atomizing air pressure of 2.5 bar. The inlet air temperature was 60° C., the outlet air temperature was 45° C., the flap was 80 m³/hr, and the spray rate was 10 g/min. After the drug-coated pellets were coated with the separating layer suspension, they were dried in the fluid bed dryer for an additional 5 minutes at 40° C. to form sub-coated pellets.

Part IV—Enteric Layer

EUDRAGIT® L30D55 methacrylic acid copolymer dispersion and triethyl citrate were mixed in a mixer for 15 minutes to form a 30 percent solution. Talc was mixed in purified water in a homogenizer for 30 minutes, and the resulting mixture of talc and water was added to the solution in the mixer. The resulting mixture was mixed for 15 minutes.

The resulting suspension was sieved, and then sprayed onto the sub-coated pellets in the fluid bed dryer. The suspension was sprayed while mixing through a 1.2 mm nozzle at an atomizing air pressure of 2.5 bar. The inlet air temperature was 38° C., the outlet air temperature was 28° C., the flap was 85 m³/hr, and the spray rate was 10 g/min. After the drug-coated pellets were coated with the separating layer suspension, they were dried in the fluid bed dryer for an additional 120 minutes at 40° C. to form enteric-coated pellets.

Part V—Finish Layer

Hypromellose, colloidal silicon dioxide and titanium dioxide were mixed in purified water in a mixer for 30 minutes to form a solution. Talc was mixed in purified water in a homogenizer for 30 minutes. The mixture of talc and water was then added to the solution in the mixer, and mixed for 15 minutes.

The resulting suspension was sieved and then sprayed onto the enteric-coated pellets in the fluid bed dryer. Spraying was accomplished with 1.2 mm nozzle and at an atomizing air pressure of 2.3 bar over a period of 60 minutes. The inlet air temperature was 55° C., the outlet air temperature was 40° C., the flap was 80 m³/hr, and the spray rate was 10 g/min. After the drug-coated pellets were coated with the separating layer suspension, they were dried in the fluid bed dryer for an additional 5 minutes at 40° C. The coated pellets were then filled into capsules.

The ingredients of the formulation of Example 1 and their function in the formulation are summarized in Table 1 below, where all concentrations are in percent by weight.

TABLE 1
Formulation of Example 1.

| | | | |
|---|---|---|---|
| | Concentration | A Preferred | Weight % of |
| Ingredient | (% w/w) | Function | Formulation |

| | |
|---|---|
| Part I - Core | 37.94% |

| | | |
|---|---|---|
| Sugar spheres | 37.94 | Capsules diluent |
| (850-1000 microns) | | |

| | |
|---|---|
| Part II - Drug Layer | 30.87% |

| | | |
|---|---|---|
| Duloxetine HCl | 19.79 | Active material |
| Sucrose NF | 2.54 | Binder |
| Povidone | 5.7 | Binder |
| Collidal Silicon | 1.04 | Glidant |
| Dioxide | | |
| Hypromellose | 1.80 | Coating agent |
| Purified Water | 80.0 | Coating solution |
| Alcohol 95.0% | 20.0 | Coating solution |

| | |
|---|---|
| Part III - Separating Layer | 16.77% |

| | | |
|---|---|---|
| OPADRY ® White | 5.68 | Coating agent |
| 39A28677 | | |
| Sucrose | 3.44 | Diluent |
| Talc | 7.18 | Thickness agent |
| Hypromellose | 0.47 | Coating agent |
| Purified water | 100.0 | Coating solution |

| | |
|---|---|
| Part IV - Enteric Layer | 12.65% |

| | | |
|---|---|---|
| EUDRAGIT ® | 8.04 | Film former |
| L30D55 (Methacrylic | | |
| acid copolymer | | |
| Dispersion) | | |
| Talc | 3.22 | Glidant |
| Triethyl Citrate | 1.39 | Plasticizer |
| Purified Water | 100.0 | Coating solution |

| | |
|---|---|
| Part V - Finish Layer | 1.77% |

| | | |
|---|---|---|
| Hypromellose | 0.70 | Coating agent |
| Talc | 0.79 | Thickening agent |
| Titanium dioxide | 0.04 | Coloring agent |
| Collidal Silicon | 0.24 | Glidant |
| Dioxide | | |
| Purified Water | 100.0 | Coating solution |
| Total Fill Weight | 100% | |

In the formulation of Example 1, the weight ratio of core:drug layer is 1.23:1; the weight ratio of drug layer:separating layer is 1.84:1; the weight ratio of separating layer:enteric layer is 1.33:1; the weight ratio of enteric layer:finish layer is 7.15:1.

### Example 2

### Preparation of a Duloxetine Hydrochloride Delayed Release Capsule Containing an Enteric Layer of Methacrylic Acid Co-Polymer

Part I—Core

Sugar spheres were obtained, and placed in a fluid bed dryer. The average diameter of the sugar spheres was 850-1000 microns.

Part II—Drug Layer

A solution of 80 percent purified water and 20 percent ethanol was prepared, and added to a mixer. Sucrose, povidone, duloxetine hydrochloride, colloidal silicon dioxide, and hypromellose were then added to the mixer, and mixed with the water and ethanol until the solids were fully dissolved.

The resulting solution was sprayed, while mixing, onto the sugar spheres in the fluid bed dryer through a 1 mm nozzle at an atomizing air pressure of 2.5 bar over a period of 240 minutes. The inlet air temperature was 60° C., the outlet air temperature was 48° C., the flap was 100 m³/hr, and the spray rate was 5 to 10 g/min. The coated sugar spheres were then dried in the fluid bed dryer for an additional 5 minutes at 40° C. to form drug-coated pellets.

Part III—Separating Layer

Sucrose, OPADRY® 39A28677, and hypromellose were mixed in purified water in a mixer until fully dissolved to form a solution. Talc was mixed in purified water in a homogenizer for 30 minutes, and the resulting mixture of talc and water was added to the solution in the mixer. The resulting mixture was mixed for 15 minutes.

The resulting suspension was sieved, and then sprayed onto the drug-coated pellets in the fluid bed dryer. The suspension was sprayed through a 1.2 mm nozzle at an atomizing air pressure of 2.5 bar over a period of 90 minutes. The inlet air temperature was 60° C., the outlet air temperature was 45° C., the flap was 80 m³/hr, and the spray rate was 10 g/min. After the drug-coated pellets were coated with the separating layer suspension, they were dried in the fluid bed dryer for an additional 5 minutes at 40° C. to form sub-coated pellets.

Part IV—Enteric Layer

EUDRAGIT® L30D55 methacrylic acid copolymer dispersion and triethyl citrate were mixed in a mixer for 15 minutes to form a 30 percent solution. Talc was mixed in purified water in a homogenizer for 30 minutes, and the resulting mixture of talc and water was added to the solution in the mixer. The resulting mixture was mixed for 15 minutes.

The resulting suspension was sieved, and then sprayed onto the sub-coated pellets in the fluid bed dryer. The suspension was sprayed through a 1.2 mm nozzle at an atomizing air pressure of 2.5 bar over a period of 45 minutes. The inlet air temperature was 38° C., the outlet air temperature was 28° C., the flap was 85 m³/hr, and the spray rate was 10 g/min. After the drug-coated pellets were coated with the separating layer suspension, they were dried in the fluid bed dryer for an additional 120 minutes at 40° C. to form enteric-coated pellets.

Part V—Finish Layer

Hypromellose, colloidal silicon dioxide and titanium dioxide were mixed in purified water in a mixer for 30 minutes to form a solution. Talc was mixed in purified water in a homogenizer for 30 minutes. The mixture of talc and water was then added to the solution in the mixer, and mixed for 15 minutes.

The resulting suspension was sieved and then sprayed onto the enteric-coated pellets in the fluid bed dryer. Spraying was accomplished with 1.2 mm nozzle and at an atomizing air pressure of 2.3 bar over a period of 60 minutes. The inlet air temperature was 55° C., the outlet air temperature was 40° C., the flap was 80 m³/hr, and the spray rate was 10 g/min. After the drug-coated pellets were coated with the separating layer suspension, they were dried in the fluid bed dryer for an additional 5 minutes at 40° C. The coated pellets were then filled into capsules to form 4000 capsules.

The ingredients of the formulation of Example 2 and their function in the formulation are summarized in Table 2 below, where all concentrations are in percent by weight.

TABLE 2
Formulation of Example 2.

| | | | |
|---|---|---|---|
| | Concentration | A Preferred | Weight % of |
| Ingredient | (% w/w) | Function | Formulation |

| | |
|---|---|
| Part I - Core | 37.94% |

| | | |
|---|---|---|
| Sugar spheres | 37.94 | Capsules diluent |
| (850-1000 microns) | | |

Part II - Drug Layer

| | | |
|---|---|---|
| Duloxetine HCl | 19.79 | Active material |
| Sucrose NF | 2.54 | Binder |
| Povidone | 5.7 | Binder |
| Collidal Silicon | 1.04 | Glidant |
| Dioxide | | |
| Hypromellose | 1.80 | Coating agent |
| Purified Water | 80.0 | Coating solution |
| Alcohol 95.0% | 20.0 | Coating solution |

| | |
|---|---|
| Part III - Separating Layer | 30.87% |

| | | |
|---|---|---|
| OPADRY ® White | 5.68 | Coating agent |
| 39A28677 | | |
| Sucrose | 3.44 | Diluent |
| Talc | 7.18 | Thickness agent |
| Hypromellose | 0.47 | Coating agent |
| Purified water | 100.0 | Coating solution |

| | |
|---|---|
| Part IV - Enteric Layer | 16.77% |

| | | |
|---|---|---|
| EUDRAGIT ® | 8.04 | Film former |
| L30D55 (Methacrylic | | |
| acid copolymer | | |
| Dispersion) | | |
| Talc | 3.22 | Glidant |
| Triethyl Citrate | 1.39 | Plasticizer |
| Purified Water | 100.0 | Coating solution |

| | |
|---|---|
| Part V - Finish Layer | 1.77% |

| | | |
|---|---|---|
| Hypromellose | 0.70 | Coating agent |
| Talc | 0.79 | Thickening agent |
| Titanium dioxide | 0.04 | Coloring agent |
| Collidal Silicon | 0.24 | Glidant |
| Dioxide | | |
| Purified Water | 100.0 | Coating solution |
| Total Fill Weight | 100% | |

In the formulation of Example 2, the weight ratio of core:drug layer is 1.23:1; the weight ratio of drug layer:separating layer is 1.84:1; the weight ratio of separating layer:enteric layer is 1.33:1; the weight ratio of enteric layer:finish layer is 7.15:1.

### Example 3

### Preparation of a Duloxetine Hydrochloride Delayed Release Capsule Containing an Enteric Layer of Hydroxypropyl Methycellulose Phthalate

Part I—Core

Sugar spheres are obtained, and placed in a fluid bed dryer. The average diameter of the sugar spheres is 850-1000 microns.

Part II—Drug Layer

A solution of 75-90 percent purified water and 10-30 percent ethanol is prepared, and added to a mixer. Sucrose, povidone, duloxetine hydrochloride, colloidal silicon dioxide, and hypromellose are then added to the mixer, and mixed with the water and ethanol until the solids are fully dissolved.

The resulting solution is sprayed, while mixing, onto the sugar spheres in the fluid bed dryer through a l mm nozzle at an atomizing air pressure of 2.5 bar over a period of 240 minutes. The inlet air temperature is 60° C., the outlet air temperature is 48° C., the flap is 100 m³/hr, and the spray rate is 5 to 10 g/min. The coated sugar spheres are then dried in the fluid bed dryer for an additional 5 minutes at 40° C. to form drug-coated pellets.

Part III—Separating Layer

Sucrose, OPADRY® 39A28677, and hypromellose are mixed in purified water in a mixer until fully dissolved to form a solution. Talc is mixed in purified water in a homogenizer for 30 minutes, and the resulting mixture of talc and water is added to the solution in the mixer. The resulting mixture is mixed for 15 minutes.

The resulting suspension is sieved, and then sprayed onto the drug-coated pellets in the fluid bed dryer. The suspension is sprayed through a 1.2 mm nozzle at an atomizing air pressure of 2.5 bar over a period of 90 minutes. The inlet air temperature is 60° C., the outlet air temperature is 45° C., the flap is 80 m³/hr, and the spray rate is 10 g/min. After the drug-coated pellets are coated with the separating layer suspension, they are dried in the fluid bed dryer for an additional 5 minutes at 40° C. to form sub-coated pellets.

Part IV—Enteric Layer

HPMCP H-55 (Hydroxypropyl Methycellulose Phthalate) is dissolved in a solvent system of ethanol/purified water (80:20 w/w%) at a temperature of not less than 25° C. to form a 5-7% percent solution of HPMCP. Triethyl citrate is then added to the solution and the solution is mixed for 15 minutes to form a solution having 8% by weight triethyl citrate relative to the amount of HPMCP. Talc is mixed in purified water in a homogenizer for 30 minutes, and the resulting mixture of talc and water is added to the solution in the mixer to form a mixture having talc in an amount of 37% by weight relative to the amount of HPMCP. The resulting mixture is then mixed for 15 minutes.

The resulting suspension is sieved, and then sprayed onto the sub-coated pellets in the fluid bed dryer. The suspension is sprayed through a 1.0 mm nozzle at an atomizing air pressure of 2.5 bar over a period of 180 minutes. The inlet air temperature is 45° C.-55° C., the outlet air temperature is 30° C.-40° C., the flap is 80-100 m³/hr, and the spray rate is 4-20 g/min.

Part V—Finish Layer

Hypromellose, colloidal silicon dioxide and titanium dioxide are mixed in purified water in a mixer for 30 minutes to form a solution. Talc is mixed in purified water in a homogenizer for 30 minutes. The mixture of talc and water is then added to the solution in the mixer, and mixed for 15 minutes.

The resulting suspension is sieved and then sprayed onto the enteric-coated pellets in the fluid bed dryer. Spraying is accomplished with a 1.2 mm nozzle and at an atomizing air pressure of 2.3 bar over a period of 60 minutes. The inlet air temperature is 55° C., the outlet air temperature is 40° C., the flap is 80 m³/hr, and the spray rate is 10 g/min. After the drug-coated pellets are coated with the separating layer suspension, they are dried in the fluid bed dryer for an additional 5 minutes at 40° C. The coated pellets are then filled into capsules.

The ingredients of the formulation of Example 3 and their function in the formulation are summarized in Table 3 below, where all concentrations are in percent by weight.

TABLE 3
Formulation of Example 3.

| | Concentration | A Preferred | Weight % of |
|---|---|---|---|
| Ingredient | (% w/w) | Function | Formulation |
| Part I - Core | | | 37.94% |
| Sugar spheres | 37.94 | Capsules diluent | |
| (850-1000 microns) | | | |

| | |
|---|---|
| Part II - Drug Layer | 30.87% |

| | | |
|---|---|---|
| Duloxetine HCl | 19.79 | Active material |
| Sucrose NF | 2.54 | Binder |
| Povidone | 5.7 | Binder |
| Collidal Silicon | 1.04 | Glidant |
| Dioxide | | |
| Hypromellose | 1.80 | Coating agent |
| Purified Water | 80.0 | Coating solution |
| Alcohol 95.0% | 20.0 | Coating solution |

| | |
|---|---|
| Part III - Separating Layer | 16.77% |

| | | |
|---|---|---|
| OPADRY ® | 5.68 | Coating agent |
| White 39A28677 | | |
| Sucrose | 3.44 | Diluent |
| Talc | 7.18 | Thickness agent |
| Hypromellose | 0.47 | Coating agent |
| Purified water | 100.0 | Coating solution |

| | |
|---|---|
| Part IV - Enteric Layer | 12.65% |

| | | |
|---|---|---|
| HPMCP HP-55 | 8.04 | Film former |
| (Hydroxypropyl | | |
| Methylcellulose | | |
| Phthalate) | | |
| Talc | 3.22 | Glidant |
| Triethyl Citrate | 1.39 | Plasticizer |
| Ethanol 95% | 80.0 | Coating solvent |
| Purified Water | 20.0 | Coating solvent |

| | |
|---|---|
| Part V - Finish Layer | 1.77% |

| | | |
|---|---|---|
| Hypromellose | 0.70 | Coating agent |
| Talc | 0.79 | Thickening agent |
| Titanium dioxide | 0.04 | Coloring agent |
| Collidal Silicon | 0.24 | Glidant |
| Dioxide | | |
| Purified Water | 100.0 | Coating solution |
| Total Fill Weight | 100% | |

In the formulation of Example 3, the weight ratio of core:drug layer is 1.23:1; the weight ratio of drug layer:separating layer is 1.84:1; the weight ratio of separating layer:enteric layer is 1.33:1; the weight ratio of enteric layer:finish layer is 7.15:1.

### Example 4

### Preparation of a Duloxetine Hydrochloride Delayed Release Capsule Containing an Enteric Layer of Hydroxypropyl Methycellulose Phthalate

Part I—Core

CELLETS® microcrystalline cellulose pellets are obtained, and placed in a fluid bed dryer. The average diameter of the CELLETS® is 500-710 microns.

Part II—Drug Layer

A solution of 75-90 percent purified water and 10-30 percent ethanol is prepared, and added to a mixer. Sucrose, povidone, duloxetine hydrochloride, colloidal silicon dioxide, and hypromellose are then added to the mixer, and mixed with the water and ethanol until the solids are fully dissolved.

The resulting solution is sprayed, while mixing, onto the sugar spheres in the fluid bed dryer through a 1 mm nozzle at an atomizing air pressure of 2.5 bar over a period of 240 minutes. The inlet air temperature is 60° C., the outlet air temperature is 48° C., the flap is 100 m³/hr, and the spray rate is 5 to 10 g/min. The coated sugar spheres are then dried in the fluid bed dryer for an additional 5 minutes at 40° C. to form drug-coated pellets.

Part III—Separating Layer

Sucrose, OPADRY® 39A28677, and hypromellose are mixed in purified water in a mixer until fully dissolved to form a solution. Talc is mixed in purified water in a homogenizer for 30 minutes, and the resulting mixture of talc and water is added to the solution in the mixer. The resulting mixture is mixed for 15 minutes.

The resulting suspension is sieved, and then sprayed onto the drug-coated pellets in the fluid bed dryer. The suspension is sprayed through a 1.2 mm nozzle at an atomizing air pressure of 2.5 bar over a period of 90 minutes. The inlet air temperature is 60° C., the outlet air temperature is 45° C., the flap is 80 m³/hr, and the spray rate is 10 g/min. After the drug-coated pellets are coated with the separating layer suspension, they are dried in the fluid bed dryer for an additional 5 minutes at 40° C. to form sub-coated pellets.

Part IV—Enteric Layer

HPMCP H-55 (Hydroxypropyl Methycellulose Phthalate) is dissolved in a solvent system of ethanol/purified water (80:20 w/w%) at a temperature of not less than 25° C. to form a 5-7% percent solution of HPMCP. Triethyl citrate is then added to the solution and the solution is mixed for 15 minutes to form a solution having 8% by weight triethyl citrate relative to the amount of HPMCP. Talc is mixed in purified water in a homogenizer for 30 minutes, and the resulting mixture of talc and water is added to the solution in the mixer to form a mixture having talc in an amount of 37% by weight relative to the amount of HPMCP. The resulting mixture is then mixed for 15 minutes.

The resulting suspension is sieved, and then sprayed onto the sub-coated pellets in the fluid bed dryer. The suspension is sprayed through a 1.0 mm nozzle at an atomizing air pressure of 2.5 bar over a period of 180 minutes. The inlet air temperature is 45° C.-55° C., the outlet air temperature is 30° C.-40° C., the flap is 80-100 m³/hr, and the spray rate is 4-20 g/min.

Part V—Finish Layer

Hypromellose, colloidal silicon dioxide and titanium dioxide are mixed in purified water in a mixer for 30 minutes to form a solution. Talc is mixed in purified water in a homogenizer for 30 minutes. The mixture of talc and water is then added to the solution in the mixer, and mixed for 15 minutes.

The resulting suspension is sieved and then sprayed onto the enteric-coated pellets in the fluid bed dryer. Spraying is accomplished with a 1.2 mm nozzle and at an atomizing air pressure of 2.3 bar over a period of 60 minutes. The inlet air temperature is 55° C., the outlet air temperature is 40° C., the flap is 80 m³/hr, and the spray rate is 10 g/min. After the drug-coated pellets are coated with the separating layer suspension, they are dried in the fluid bed dryer for an additional 5 minutes at 40° C. The coated pellets are then filled into capsules.

The ingredients of the formulation of Example 4 and their function in the formulation are summarized in Table 4 below, where all concentrations are in percent by weight.

TABLE 4
Formulation of Example 4.

| | Concentration | A Preferred | Weight % of |
|---|---|---|---|
| Ingredient | (% w/w) | Function | Formulation |
| Part I - Core | | | 27.64% |
| CELLETS ® | 27.64 | Capsules diluent | |
| (500-710 microns) | | | |

| | |
|---|---|
| Part II - Drug Layer | 21.86% |

| | | |
|---|---|---|
| Duloxetine HCl | 16.91 | Active material |
| Sucrose NF | N.A | Binder |
| Talc | 2.01 | Thickness agent |
| Povidone | 1.51 | Binder |
| Collidal Silicon | 1.43 | Glidant |
| Dioxide | | |
| Hypromellose | N.A | Coating agent |
| Purified Water | 85.0 | Coating solution |
| Alcohol 95.0% | 15.0 | Coating solution |

| | |
|---|---|
| Part III - Separating Layer | 35.18% |

| | | |
|---|---|---|
| OPADRY ® | 14.83 | Coating agent |
| White 39A28677 | | |
| Sucrose | 4.77 | Diluent |
| Talc | 14.83 | Thickness agent |
| Hypromellose | 0.75 | Coating agent |
| Purified water | 100.0 | Coating solution |

| | |
|---|---|
| Part IV - Enteric Layer | 15.32% |

| | | |
|---|---|---|
| HPMCP HP-55 | 10.55 | Film former |
| (Hydroxypropyl | | |
| Methylcellulose | | |
| Phthalate) | | |
| Talc | 3.92 | Glidant |
| Triethyl Citrate | 0.85 | Plasticizer |
| Ethanol 95% | 80.0 | Coating solvent |
| Purified Water | 20.0 | Coating solvent |

In the formulation of Example 4, the weight ratio of core:drug layer is 1.26:1; the weight ratio of drug layer:separating layer is 0.62:1; and the weight ratio of separating layer:enteric layer is 2.30:1.

### Example 5

### Preparation of a Duloxetine Hydrochloride Delayed Release Capsule with an Enteric Layer of Methacrylic Acid Co-Polymer

Part I—Core

Sugar spheres were obtained, and placed in a fluid bed dryer. The average diameter of the sugar spheres was 850-1000 microns.

Part II—Drug Layer

A solution of 85 percent purified water and 15 percent ethanol was prepared, and added to a mixer. Sucrose, povidone, duloxetine hydrochloride, and colloidal silicon dioxide were then added to the mixer, and mixed with the water and ethanol until the solids were fully dissolved to form a solution. Talc was mixed in purified water in a Silverson homogenizer for 30 minutes, and the resulting mixture of talc and water was added to the solution in the mixer. The resulting mixture was mixed for 15 minutes.

The resulting mixture was sieved and then sprayed, while mixing, onto the sugar spheres in the fluid bed dryer through a 1.2 mm nozzle at an atomizing air pressure of 2.5 bar over a period of 240 minutes. The inlet air temperature was 60° C., the outlet air temperature was 48° C., the flap was 100 m³/hr, and the spray rate was 5 to 10 g/min. The coated sugar spheres were then dried in the fluid bed dryer for an additional 5 minutes at 40° C. to form drug-coated pellets.

Part III—Separating Layer

Sucrose, OPADRY® 39A28677, and hypromellose were mixed in purified water in a mixer until fully dissolved to form a solution. Talc was mixed in purified water in a homogenizer for 30 minutes, and the resulting mixture of talc and water was added to the solution in the mixer. The resulting mixture was mixed for 15 minutes.

The resulting suspension was sieved, and then sprayed onto the drug-coated pellets in the fluid bed dryer. The suspension was sprayed through a 1.2 mm nozzle at an atomizing air pressure of 2.5 bar over a period of 90 minutes. The inlet air temperature was 60° C., the outlet air temperature was 45° C., the flap was 80 m³/hr, and the spray rate was 10 g/min. After the drug-coated pellets were coated with the separating layer suspension, they were dried in the fluid bed dryer for an additional 5 minutes at 40° C. to form sub-coated pellets.

Part IV—Enteric Layer

EUDRAGIT® L30D55 methacrylic acid copolymer dispersion and triethyl citrate were mixed in a mixer for 15 minutes to form a 25-30 percent solution of film coating. Talc was mixed in purified water in a homogenizer for 30 minutes, and the resulting mixture of talc and water was added to the solution in the mixer. The resulting mixture was mixed for 15 minutes.

The resulting suspension was sieved, and then sprayed onto the sub-coated pellets in the fluid bed dryer. The suspension was sprayed through a 1.2 mm nozzle at an atomizing air pressure of 2.5 bar over a period of 45 minutes. The inlet air temperature was 38° C., the outlet air temperature was 28° C., the flap was 85 m³/hr, and the spray rate was 10 g/min. After the drug-coated pellets were coated with the separating layer suspension, they were dried in the fluid bed dryer for an additional 120 minutes at 40° C. to form enteric-coated pellets.

Part V—Finish Layer

Hypromellose, colloidal silicon dioxide and titanium dioxide were mixed in purified water in a mixer for 30 minutes to form a solution. Talc was mixed in purified water in a homogenizer for 30 minutes. The mixture of talc and water was then added to the solution in the mixer, and mixed for 15 minutes.

The resulting suspension was sieved and then sprayed onto the enteric-coated pellets in the fluid bed dryer. Spraying was accomplished with 1.2 mm nozzle and at an atomizing air pressure of 2.3 bar over a period of 60 minutes. The inlet air temperature was 55° C., the outlet air temperature was 40° C., the flap was 80 m³/hr, and the spray rate was 10 g/min. After the drug-coated pellets were coated with the separating layer suspension, they were dried in the fluid bed dryer for an additional 5 minutes at 40° C. The coated pellets were then filled into capsules to form 4000 capsules.

The ingredients of the formulation of Example 5 and their function in the formulation are summarized in Table 5 below, where all concentrations are in percent by weight.

TABLE 5
Formulation of Example 5.

| | Concentration | A Preferred | Weight % of |
|---|---|---|---|
| Ingredient | (% w/w) | Function | Formulation |
| Part I - Core | | | 40.4% |
| Sugar spheres | 40.4 | Capsules diluent | |
| (850-1000 microns) | | | |

| | |
|---|---|
| Part II - Drug Layer | 17.58% |

| | | |
|---|---|---|
| Duloxetine HCl | 13.60 | Active material |
| Povidone | 1.21 | Binder |
| Collidal Silicon | 1.15 | Glidant |
| Dioxide | | |
| Talc USP extra fine | 1.62 | Coating agent |
| Purified Water | 85.0 | Coating solution |
| Alcohol 95.0% | 15.0 | Coating solution |

| | |
|---|---|
| Part III - Separating Layer | 17.36% |

| | | |
|---|---|---|
| OPADRY ® | 7.27 | Coating agent |
| White 39A28677 | | |
| Sucrose | 2.42 | Diluent |
| Talc USP extra fine | 7.27 | Thickness agent |
| Hypromellose 606 | 0.4 | Coating agent |
| Purified water | 100.0 | Coating solution |

| | |
|---|---|
| Part IV - Enteric Layer | 22.62% |

| | | |
|---|---|---|
| EUDRAGIT ® | 14.28 | Film former |
| L30D55 (Methacrylic | | |
| acid copolymer | | |
| Dispersion) | | |
| Talc USP extra fine | 5.76 | Glidant |
| Triethyl Citrate | 2.58 | Plasticizer |
| Purified Water | 100.0 | Coating solution |

| | |
|---|---|
| Part V - Finish Layer | 2.04% |

| | | |
|---|---|---|
| Hypromellose 603 | 0.81 | Coating agent |
| Talc USP extra fine | 0.89 | Thickening agent |
| Titanium dioxide | 0.05 | Coloring agent |
| Collidal Silicon | 0.29 | Glidant |
| Dioxide | | |
| Purified Water | 100.0 | Coating solution |
| Total Fill Weight | 100% | |

In the formulation of Example 5, the weight ratio of core:drug layer is 2.30:1; the weight ratio of drug layer:separating layer is 1.01:1; the weight ratio of separating layer:enteric layer is 0.77:1; the weight ratio of enteric layer:finish layer is 11.09:1.

### Example 6

### Stability of Duloxetine Hydrochloride Delayed Release Capsules Upon Storage

a. Duloxetine hydrochloride delayed release capsules containing an enteric layer of methacrylic acid co-polymer

Capsules having the formulation listed in Table 6 were packed in containers with aluminium heat induction liner and a child resistant (clic-loc) 38 mm plastic cap manufactured by Owens Brockway Plastics and stored at 40° C. (±2° C.) and 75% (±5%) relative humidity for 2 months.

TABLE 6
Formulation of duloxetine hydrochloride delayed release capsules
containing an enteric layer of methacrylic acid co-polymer

| Ingredient | Amount per capsule | |
|---|---|---|
| Duloxetine hydrochloride | 67.3 | mg |
| Sugar spheres (850-1000 microns) | 150.0 | mg |
| Hypromellose USP (PHARMACOAT ® 606) | 1.6 | mg |
| Povidone USP (PVP K-30) | 6.0 | mg |
| Colloidal silicon dioxide NF (AEROSIL ® 200) | 5.7 | mg |
| Sucrose NF | 11.7 | mg |
| OPADRY ® 39A28677 white | 19.3 | mg |
| Talc USP extra fine | 43.35 | mg |
| Methacrylic acid co-polymer dispersion NF | 27.34 | mg |
| (EUDRAGIT ® L30 D55) | | |
| Triethyl citrate NF | 4.71 | mg |

The capsules were analyzed by HPLC at time zero, after one month of storage, and after two months of storage to determine the presence and amount of duloxetine hydrochloride impurities. The results are shown in Table 7. The percentages in Table 7 are expressed in terms of % area by HPLC based upon a duloxetine hydrochloride standard.

TABLE 7
Storage stability of duloxetine hydrochloride delayed release
capsules containing an enteric layer of methacrylic acid co-polymer

| Duloxetine | Storage Time | | | |
|---|---|---|---|---|
| Hydrochloride Impurity | Time Zero | 1 month | 2 months | 3 months |
| 1-Naphthol | <0.05% | 0.05% | 0.05% | 0.08% |
| (+)-N-methyl-3-(1- | 0.08% | 0.08% | 0.08% | 0.08% |
| naphtalenyloxy)-3-(3- | | | | |
| thienyl)propanamine | | | | |
| (“DLX-ISO3”) | | | | |
| Total impurities | 0.13% | 0.13% | 0.13% | 0.16% |

*DLX-ISO3 has a relative retention time of 1.04 and 1-Naphthol has a relative retention time of 1.3.

b. CYMBALTA® duloxetine hydrochloride delayed release capsules containing an enteric layer of HPMCAS

CYMBALTA® 60 mg delayed release capsules having the formulation listed in Table 8 were stored in their original packaging (i.e., a high density polyethylene (HDPE) bottle with a child resistant cap (CRC), induction sealed) at 40° C. (±2° C.) and 75% (±5%) relative humidity for 3 months.

TABLE 8
Formulation of CYMBALTA ® duloxetine hydrochloride delayed
release capsules containing an enteric layer of HPMCAS
Ingredient

Duloxetine hydrochloride
Sugar spheres
Hypromellose
Sodium lauryl sulfate
Colloidal silicon dioxide
Sucrose
Titanium dioxide
Talc
HPMCAS
Triethyl citrate NF
Gelatin
FD&C blue No. 2
Iron oxide yellow

The capsules were analyzed by HPLC at time zero and after three months of storage to determine the presence and amount of duloxetine hydrochloride impurities. The results are shown in Table 9. The percentages in Table 9 are expressed in terms of % area by HPLC.

TABLE 9
Storage stability of CYMBALTA ® duloxetine hydrochloride delayed
release capsules containing an enteric layer of HPMCAS

| | |
|---|---|
| Duloxetine | Storage Time |

| Hydrochloride Impurity | Time Zero | 3 months |
|---|---|---|
| 1-Naphthol | 0.12% | 0.06% |
| DLX-ISO3 | 0.06% | 0.10% |
| Total impurities | 0.18% | 0.16% |

*DLX-ISO3 has a relative retention time of 1.04 and 1-Naphthol has a relative retention time of 1.29.

While it is apparent that the invention disclosed herein is well calculated to fulfill the objects stated above, it will be appreciated that numerous modifications and embodiments may be devised by those skilled in the art. Therefore, it is intended that the appended claims cover all such modifications and embodiments as falling within the true spirit and scope of the present invention.

1. A duloxetine hydrochloride delayed release formulation, comprising:
(a) an inert core;
(b) a drug layer comprising duloxetine hydrochloride;
(c) a separating layer; and
(d) an enteric layer comprising at least one of methacrylic acid copolymer and hydroxypropyl methyl cellulose phthalate.

2. The formulation of claim 1, further comprising a finish layer.

3. The formulation of claim 1, wherein the inert core comprises at least one of sugar spheres or pellets of microcrystalline cellulose.

4. The formulation of claim 1, wherein the core is present in a weight ratio of about 1:1 to about 2.5:1 relative to the drug layer.

5. The formulation of claim 1, wherein the drug layer further comprises at least one pharmaceutically acceptable excipient selected from binders, glidants, coating agents, and anti-static agents.

6. The formulation of claim 1, wherein the drug layer further comprises at least one pharmaceutically acceptable excipient selected from sucrose, povidone, colloidal silicon dioxide, hypromellose, and talc.

7. The formulation of claim 1, wherein the drug layer comprises duloxetine hydrochloride, sucrose, povidone, colloidal silicon dioxide, and hypromellose.

8. The formulation of claim 1, wherein the drug layer is present in an amount of about 40 percent to about 90 percent by weight of the formulation.

9. The formulation of claim 1, wherein the drug layer is present in an amount of about 50 percent to about 75 percent by weight of the formulation.

10. The formulation of claim 1, wherein the drug layer is present in a weight ratio of about 0.5:1 to about 2:1 relative to the separating layer.

11. The formulation of claim 1, wherein the separating layer comprises a coating agent.

12. The formulation of claim 11, wherein the separating layer further comprises at least one additional pharmaceutically acceptable excipient selected from diluents, anti-adherents, and thickening agents.

13. The formulation of claim 11, wherein the separating layer further comprises at least one additional pharmaceutically acceptable excipient selected from sucrose, talc, povidone, and silicon dioxide.

14. The formulation of claim 1, wherein the separating layer comprises hypromellose, titanium dioxide, iron oxide, sucrose, and talc.

15. The formulation of claim 1, wherein the separating layer is present in an amount of about 8 percent to about 60 percent by weight of the formulation.

16. The formulation of claim 1, wherein the separating layer is present in an amount of about 15 percent to about 45 percent by weight of the formulation.

17. The formulation of claim 1, wherein the separating layer is present in a weight ratio of about 0.5:1 to about 3:1 relative to the enteric layer.

18. The formulation of claim 1, wherein the enteric layer further comprises at least one pharmaceutically acceptable excipient selected from glidants and plasticizers.

19. The formulation of claim 1, wherein the enteric layer further comprises at least one pharmaceutically acceptable excipient selected from talc and triethyl citrate.

20. The formulation of claim 1, wherein the enteric layer is present in an amount of about 5 percent to about 40 percent by weight of the formulation.

21. The formulation of claim 1, wherein the enteric layer is present in an amount of about 10 percent to about 30 percent by weight of the formulation.

22. The formulation of claim 2, wherein the enteric layer is present in a weight ratio of about 6:1 to about 12:1 relative to the finish layer.

23. The formulation of claim 2, wherein the finish layer comprises a coating agent.

24. The formulation of claim 2, wherein the finish layer comprises hypromellose, talc, colloidal silicon dioxide, and titanium dioxide.

25. The formulation of claim 2, wherein the finish layer is present in an amount of about 1 percent to about 15 percent by weight of the formulation.

26. A process for preparing the formulation of claim 1, comprising coating the core in succession with the drug layer, the separating layer, and then the enteric layer.

27. A process for preparing the formulation of claim 1, comprising:
(a) coating the inert core with a solution comprising duloxetine hydrochloride, sucrose, povidone, colloidal silicon dioxide, and hypromellose in a mixture of water and ethanol to obtain an inert core coated with drug layer;
(b) coating the inert core coated with drug layer with a suspension in water comprising hypromellose, titanium dioxide, iron oxide, sucrose, and talc to obtain an inert core coated drug layer and separating layer; and
(c) coating the inert core coated with drug layer and separating layer with a suspension in water comprising (i) at least one of methacrylic acid co-polymer and hydroxypropyl methyl cellulose phthalate, (ii) talc, and (iii) triethyl citrate to obtain the formulation of claim 1.

28. The process of claim 27, wherein (i) the inert core coated with drug layer is dried prior to step (b) and/or (ii) the inert core coated with drug layer and separating layer is dried prior to step (c).

29. A solid pharmaceutical dosage form comprising the formulation of claim 1.

30. The solid pharmaceutical dosage form of claim 29 in the form of a capsule.

31. A method of treatment of depression comprising administering the solid pharmaceutical dosage form of claim 29 to a patient in need thereof.

32. A duloxetine hydrochloride delayed release formulation, comprising:
(a) an inert core comprising sugar spheres or pellets of microcrystalline cellulose;
(b) a drug layer comprising duloxetine hydrochloride, sucrose, povidone, colloidal silicon dioxide, and hypromellose;
(c) a separating layer comprising hydroxypropyl cellulose, hypromellose, titanium oxide, iron oxide, sucrose, and talc;
(d) an enteric layer comprising methacrylic acid co-polymer, talc, and triethyl citrate; and
(e) a finish layer comprising hypromellose, talc, titanium dioxide, and colloidal silicon dioxide.

33. A duloxetine hydrochloride delayed release formulation, comprising:
(a) an inert core comprising sugar spheres or pellets of microcrystalline cellulose;
(b) a drug layer comprising duloxetine hydrochloride, sucrose, povidone, colloidal silicon dioxide, and hypromellose;
(c) a separating layer comprising hydroxypropyl cellulose, hypromellose, titanium oxide, iron oxide, sucrose, and talc;
(d) an enteric layer comprising hydroxypropyl methylcellulose phthalate, talc, and triethyl citrate; and
(e) a finish layer comprising hypromellose, talc, titanium dioxide, and colloidal silicon dioxide.

34. A duloxetine hydrochloride delayed release formulation, comprising:
(a) an inert core;
(b) a drug layer comprising duloxetine hydrochloride;
(c) a separating layer; and
(d) an enteric layer comprising at least one enteric polymer, with the proviso that the enteric polymer is not hydroxypropyl methylcellulose acetate succinate.
